(19) United States
(12) Patent Application Publication
(10) Pub. No.: US 2015/0241716 A1
(43) Pub. Date: Aug. 27, 2015

(54) PROTECTIVE LENS FOR PEOPLE WITH PRESBYOPIA

(71) Applicant: CHIN-JEN CHEN, TAINAN CITY (TW)

(72) Inventor: CHIN-JEN CHEN, TAINAN CITY (TW)

(21) Appl. No.: 14/707,291

(22) Filed: May 8, 2015

Related U.S. Application Data
(63) Continuation-in-part of application No. 13/866,230, filed on Apr. 19, 2013.

(30) Foreign Application Priority Data
May 21, 2012 (TW) 101209554

(51) Int. Cl. G02C 7/06; A61F 9/02; G02C 13/00

(52) U.S. Cl. CPC G02C 7/06; G02C 13/001; A61F 9/02; A61F 2009/021

(57) ABSTRACT

A protective lens for people with presbyopia is revealed. The protection lens includes a plano lens area and a presbyopic lens area. Both the plano lens area and the presbyopic lens area are produced by injection molding which is performed by an injection molding machine with a single material outlet. The presbyopic lens area is defined in and connected to the plano lens area. There is no overlap between the plano lens area and the presbyopic lens area. The plano lens area and the presbyopic lens area are connected directly without any medium therebetween. The presbyopic lens area is a circular area with a diameter of 3-5 centimeter. The presbyopic lens area is just corresponding to eyes on people's face after mounted to an eyeglass frame. Thereby the protective lens not only protects people's eyes during working but also allows people with presbyopia see close objects clearly.

### CROSS REFERENCE TO RELATED APPLICATION

This application is a continuation-in-part of U.S. patent application Ser. No. 13/866,230, filed on Apr. 19, 2013, currently pending, which claims the benefits of the Taiwan Patent Application Serial Number 101209554, filed on May 21, 2012, the subject matter of which is incorporated herein by reference.

### BACKGROUND OF THE INVENTION

1. Fields of the Invention

The present invention relates to a protective lens, especially to a protective lens used for eyeglasses that people with presbyopia can wear during their work.

2. Descriptions of Related Art

People have presbyopia due to aging of eyes. Thus they need to wear a corrective eyewear and a protective eyewear at the same time while working. As shown in FIG. 1, the protective eyewear 2 completely covers the corrective eyewear 1 for presbyopia and the corrective eyewear 1 for presbyopia is located in the protective eyewear 2. However, in such use mode, the user needs to wear two pairs of eyewear-the corrective eyewear 1 for presbyopia and the protective eyewear 2 at the same time. This is quite uncomfortable. While not in use, the protective eyewear 2 is taken off for storage. Thus such design is inconvenient and trouble in use and storage. Moreover, the user needs to buy both the corrective eyewear 1 for presbyopia and the protective eyewear 2 with plano lenses. Thus the cost spent on the eyewear is high.

Refer to FIG. 2, in order to solve the above problem, an eyeglass frame 5 assembled with a presbyopic lens 3 and a protective lens 4 simultaneously has been invented and provided. A first connection slot 51 and a second connection slot 52 are mounted in the eyeglass frame 5, parallel to each other. The first connection slot 51 is used to mount the presbyopic lens 3 while the second connection slot 52 is assembled with the protective lens 4. Thus the user can directly set the protective lens 4 in the eyeglass frame 5 assembled with optical lenses. He or she doesn't need to purchase another protective eyewear. A burden on the cost of the eyewear can be decreased. However, the design still has shortcomings of easy loss of components while being disassembled for storage.

Refer to U.S. Pat. No. 1,865,714, a hole is pierced in a large blank. Then a corrective lens is mounted into the hole in the blank by fusing flux. However, the processing and molding processes are complicated. The adhesion precision increases difficulty in the processing and the yield rate is reduced. There is a difference in the image due to the fusing flux. Moreover, the connection between the corrective lens and the hole is easy to be affected by thermal expansion and contraction.

Refer to U.S. Pat. No. 1,835,483, a large countersink is formed in the convex side of a blank. Then a button or disk, having a higher refractive index than the blank is fused in the countersink. Yet the melting of the corrective lens results in complicated processing procedures. The precision of the melting makes the processing and the connection more difficult. The yield rate is low. There is a difference in the image at the connection area due to different refractive index. The connection between the disk and the blank is affected by thermal expansion and contraction.

Refer to U.S. Pat. No. 6,196,678, the corrective lens and the shield of the protective glasses are not on the same plane. The corrective lenses are incorporated in the lower portion of the shield. The corrective lenses are projecting from the shield. Thus users will see an overlapped image or a difference in the image while wearing the protective glasses and this affects user's operation.

Refer to U.S. Pat. No. 6,170,952, the reading lenses are secured to the surface of a plano lens by an adhesive. The light passes the plano lens first and then the reading lenses to form an image on user's eyes. Yet different medium have different refractive index. The adhesive used affects the entrance direction of the light and causes the problem, even the reading lenses and the plano lens made from the same material. In order to overcome the above shortcomings, there is a need to provide a novel protective eyewear.

### SUMMARY OF THE INVENTION

Therefore it is a primary object of the present invention to provide a protective lens for vision correction that not only protects users' eyes but also has the function of vision correction. For users need vision correction, they feel more comfortable and convenient while wearing a pair of goggles with the protective lenses of the present invention. Moreover, the cost they spent on eyeglasses and the goggles can be reduced.

In order to achieve above object, a protective lens for vision correction according to the present invention is provided.

The protection lens includes a plano lens area and a presbyopic lens area. Both the plano lens area and the presbyopic lens area are produced by injection molding which is performed by an injection molding machine with a single material outlet. The presbyopic lens area is formed in and connected to the plano lens area. There is no overlap between the plano lens area and the presbyopic lens area. The plano lens area and the presbyopic lens area are connected directly without any medium therebetween. After being cut and disposed into an eyeglass frame, the presbyopic lens area is just located at the position corresponding to eyes on user's face.

The presbyopic lens area is a circular area with a diameter of 3-5 centimeter.

The present invention has following advantages:

1. The protective lens includes a plano lens area and a presbyopic lens area. The presbyopic lens area is just corresponding to eyes on user's face after being cut and mounted into an eyeglass frame. Thus the protective lens not only allows people with presbyopia see close objects more clearly but also protects people's eyes during their work.

2. Both the plano lens area and the presbyopic lens area of the protective lens according to the present invention are formed by an injection molding machine with a single material outlet that performs a single time injection. Thus there is no overlap and no medium between the plano lens area and the presbyopic lens area. The light transmittance will not be affected by the medium.

3. Both the plano lens area and the presbyopic lens area of the protective lens according to the present invention are formed by a single time of injection molding. The injection molding is performed by an injection molding machine with a single material outlet. There is no medium therebetween. Thus the connection between the plano lens area and the presbyopic lens area are secured to each other firmly, without being affected by thermal expansion and contraction.

4. Furthermore, the protective lens of the present invention also provides the vision correction for presbyopia. Thus there is no need for users to wear a corrective eyewear while wearing a protective eyewear. The burden on the cost of the eyewear can be reduced.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a schematic drawing showing a user wearing a corrective eyewear and a protective eyewear at the same time;

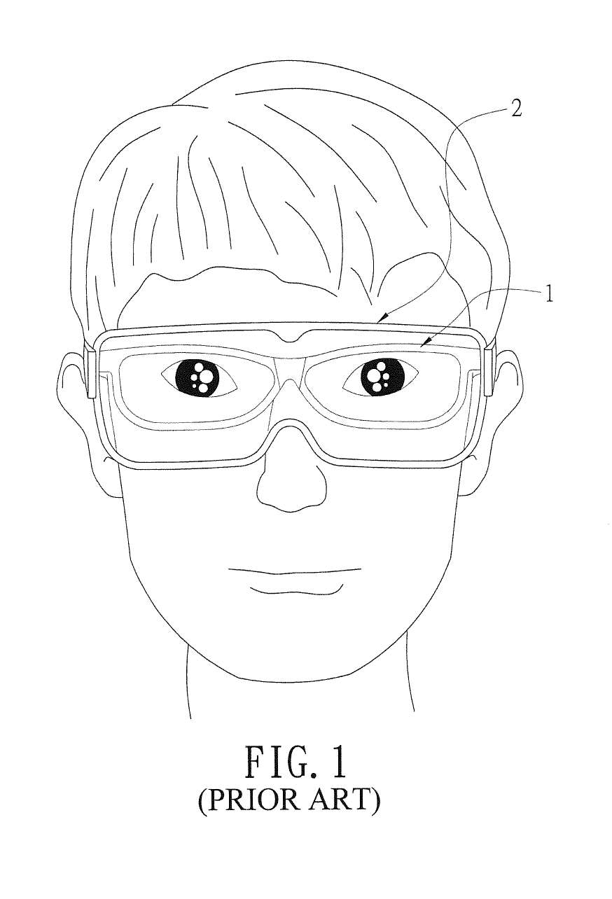

FIG. 2 is an explosive view of a conventional eyewear frame having an optical lens frame and a protective lens frame;

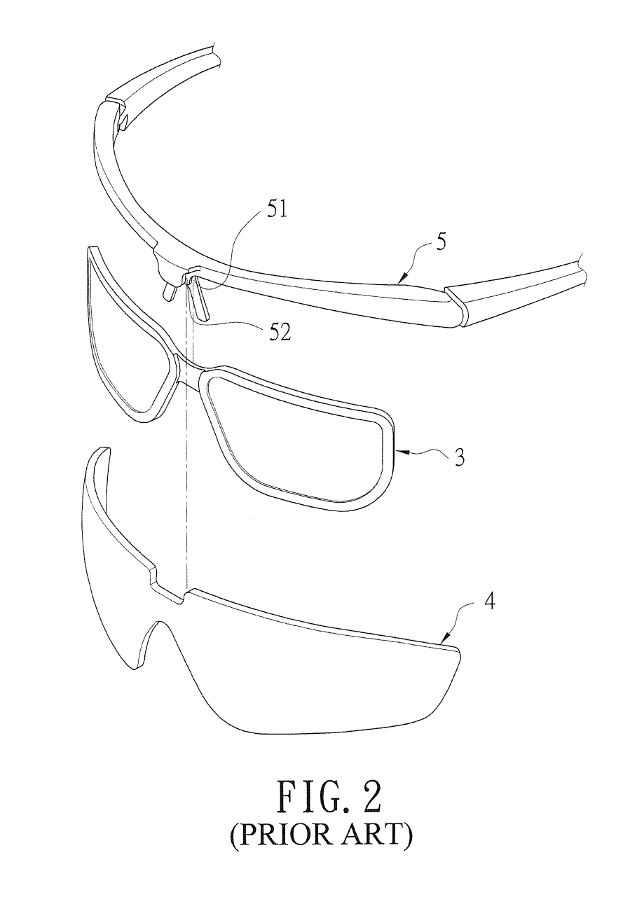

FIG. 3 is a perspective view of an embodiment of a protective lens for vision correction according to the present invention;

FIG. 4 is a perspective view of an embodiment of a protective lens for vision correction being cut and mounted in an eyeglass frame according to the present invention;

FIG. 5 is a schematic drawing showing a user wearing an eyewear assembled with protective lenses for vision correction according to the present invention.

### DETAILED DESCRIPTION OF THE PREFFERED EMBODIMENT

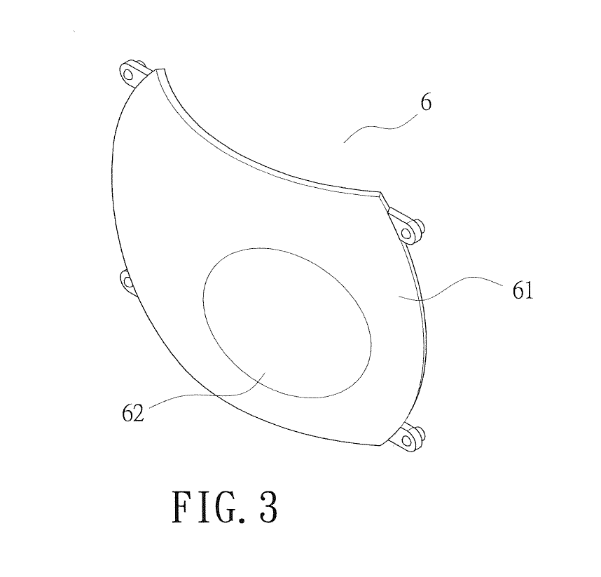

Refer to FIG. 3, a perspective view of an embodiment of a protective lens 6 for vision correction of presbyopia is revealed. The protective lens 6 includes a plano lens area 61 and a presbyopic lens area 62. Both the plano lens area 61 and the presbyopic lens area 62 are produced by injection molding, especially a single time of injection molding performed by an injection molding machine with a single material outlet. The presbyopic lens area 62 is defined to be located in and connected to the plano lens area 61. The plano lens area 61 and the presbyopic lens area 62 are connected directly without any medium therebetween. At the same time, there is no overlap between the plano lens area 61 and the presbyopic lens area 62.

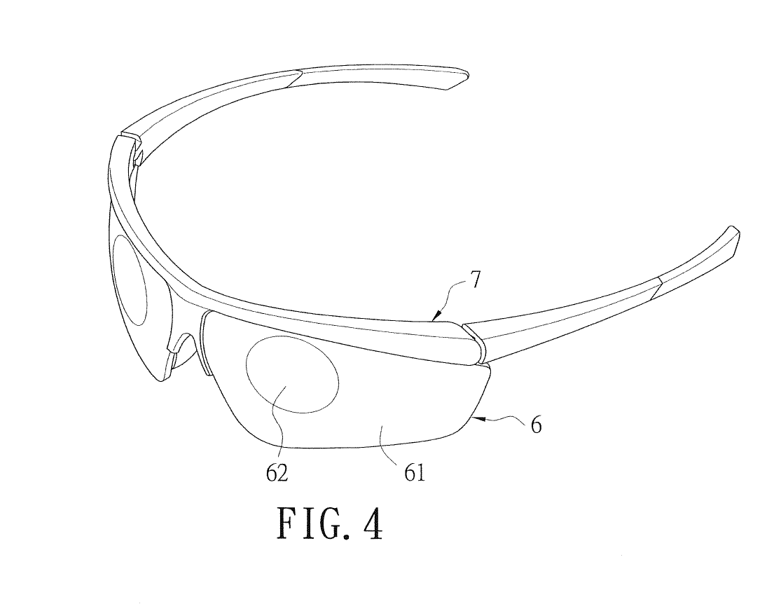

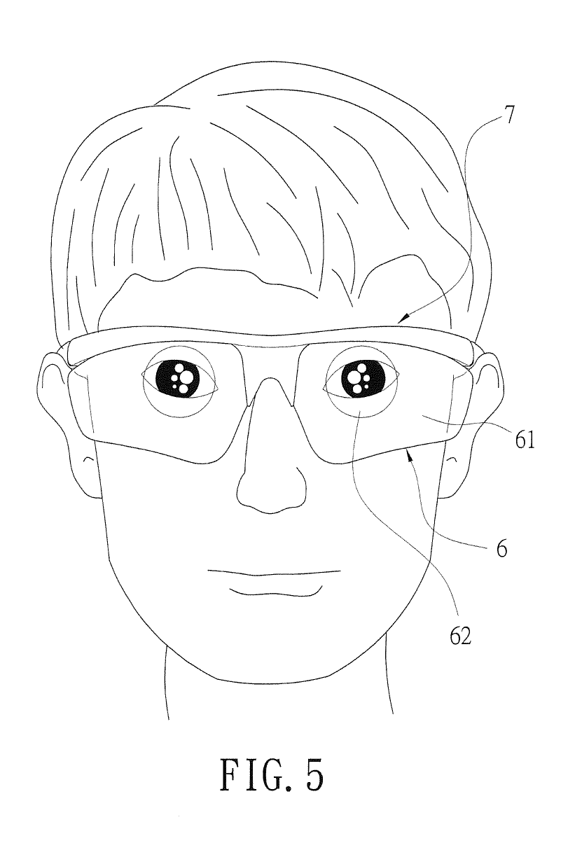

The protective lens 6 is cut according to the shape of an eyeglass frame 7 and then is assembled with the eyeglass frame 7, as shown in FIG. 4. During wearing, the presbyopic lens area 62 is just located at the position corresponding to eyes on the user's face, as shown in FIG. 5.

In a preferred embodiment of the present invention, the presbyopic lens area 62 is a circular area with a diameter of 3-5 centimeter. When people with presbyopia wear the eyeglasses with the protective lens 6, the presbyopic lens area 62 is just located at the position corresponding to eyes on user's face. Thus the presbyopic lens area 62 covers people's eyes while they are working and the line of sight moves. Therefore people with presbyopia can see close objects clearly, without affecting their operation.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A protective lens for people with presbyopia comprising a plano lens area and a presbyopic lens area;
wherein both the plano lens area and the presbyopic lens area are produced by an injection molding machine having a single material outlet that performs a single time of injection molding; the presbyopic lens area is defined to be located in and connected to the plano lens area; there is no overlap between the plano lens area and the presbyopic lens area while the plano lens area and the presbyopic lens area are connected directly without any medium therebetween; the presbyopic lens area is corresponding to eyes on people's face after being cut and mounted into an eyeglass frame.

2. The device as claimed in claim 1, wherein the presbyopic lens area is a circular area with a diameter ranging from 3 to 5 centimeter.
